(19) United States
(12) Patent Application Publication
(10) Pub. No.: US 2010/0167388 A1
(43) Pub. Date: Jul. 1, 2010

(54) Single Use Centrifuge System

(75) Inventor: Stephen B. Kessler, Princeton, MA (US)

Correspondence Address: WALKER & JOCKE, L.P.A., 231 SOUTH BROADWAY STREET, MEDINA, OH, 44256, US

(73) Assignee: PNEUMATIC SCALE CORPORATION, Cuyahoga Falls, OH (US)

(21) Appl. No.: 12/676,273

(22) PCT Filed: Apr. 21, 2009
(86) PCT No.: PCT/US2009/002464

§ 371 Date: Mar. 3, 2010

Related U.S. Application Data
(60) Provisional application No. 61/125,033, filed on Apr. 22, 2008.

(51) Int. Cl. C12M 3/00

(52) U.S. Cl. 435/308.1

(57) ABSTRACT

An improved centrifuge system having a single means for both feeding and collecting liquid streams aseptically from rotating components is provided. Also methods and apparatus for centrifugal separation of cells from cell culture media of large cell culture batches by processing a large volume within a few hours, using pre-sterilized, single-use fluid path components. The apparatus uses a sealing approach that improves reliability while avoiding air contamination as well as shedding from mechanical seals. The risk of process liquid leaks is minimized.

### BACKGROUND OF THE INVENTION

Within the field of cell culture as applied to bio-pharmaceutical processes there exists a need to separate cells from the media in which they are grown. There are several process variations currently in use which include batch, repeat batch, and perfusion. The product may be a molecular species that the cell excretes into the media, a molecular species that remains within the cell, or it may be the cell itself. In all cases the product must eventually be separated from other process components prior to final purification and product formulation and the present invention is directed to that separation in large-scale systems.

Methods currently in common usage for this purpose include continuous and semi-continuous centrifugation, tangential flow filtration (TFF) and depth filtration. The centrifuges for batch and repeat batch processes at production scale are complex systems that require clean-in-place (CIP) and steam-in-place (SIP) technology to provide an aseptic environment to prevent contamination by microorganisms. At lab scale and for perfusion processes, smaller systems are currently in use. These smaller systems are based on pre-sterilized, single-use fluid path components.

There is now a desire in the industry to use pre-sterilized, single-use centrifuges for some large, production scale operations, but the mere geometric scale-up of the existing smaller designs has not been successful beyond a processing rate of 1-2 liter/min. Technical limitations inherent in the smaller existing designs preclude a direct geometric scale-up approach. For example, simple geometric scale-up of small scale designs leads to stress levels that are unsustainable by the materials of construction typically used in single-use designs.

Another limitation that precludes simple geometric scale-up is variation in scaling of the pertinent fluid dynamic factors. For example, the maximum processing rate of any centrifuge depends on the settling velocity of the particles being separated. The settling velocity is given by a modification of Stokes' law defined by Equation 1:

$$v = \frac{Δρ \cdot {d}^{2} \cdot r \cdot {ω}^{2}}{18 \cdot μ} \qquad Equation 1$$

where ν=settling velocity, Δρ is solid-liquid density difference, d is particle diameter, r is radial position of the particle, ω is angular velocity, and μ is liquid viscosity. With respect to scale-up geometry, the radius of the bowl affects the maximum radial position r that particles can occupy. Therefore, if the other parameters in Equation 1 are held constant, an increase in bowl diameter leads to an increase in average settling velocity and improved separation efficiency. However, the effect of bowl angular velocity, ω, on settling velocity is squared. Thus, unless the scaled-up bowl can be rotated at sufficiently high angular velocity, the effect of increased bowl radius may be negated and it may not be possible to maintain the same separation efficiency on scale-up.

Since the use of pre-sterilized, single-use components is known to (i) reduce the risk of contamination, (ii) allow simplified manufacturing procedures, (iii) improve operational efficiency and (iv) lower overall costs, there is a need to develop larger scale pre-sterilized centrifuge systems.

While pre-sterilized, single-use components are currently used for media storage, mix tanks, hold tanks, and bioreactors, e.g. up to about 2000 liter capacity, there are no production size centrifuges available that employ pre-sterilized components that can be used to process such large cell culture batches. Currently, the largest semi-continuous centrifuge commercially available is only capable of processing at a maximum hydraulic rate of about 2 liters per minute (lpm), which limits its use to batch sizes of a maximum of only about 250 liters or to perfusion processes. The design of the prior art semi-continuous centrifuges limits their operating g-force to about 300×g (300 times gravity) which restricts the types of cells that can be separated and also restrains their flow rate performance. There are semi-continuous centrifuges of pre-sterilized, single-use design intended for use in other fields, such as for separating blood constituents for blood component harvesting and various therapeutic procedures. However, these devices have even lower flow rate performance.

One of the key components required in all of these systems is a means of feeding and collecting liquid streams aseptically from rotating components. U.S. Pat. Nos. 4,778,444 and 4,459,169 disclose the use of a flexible tube one end of which is attached and stationary and the other end of which rotates, i.e. a counter-rotating tube carrier continuously untwists the tube. Other commercial systems use mechanical-type rotating seals.

None of the above approaches have been successfully scaled-up to handle operating conditions inherent in a 300 to 2000 liter scale process. One aspect of this scale of operation is the need to be able to feed liquid in and out of a rotating component at flow rates ranging from about 3 to 30 liter/min. Reliability of the untwisting tube design has proved to be a limitation for long duration processes such as perfusion which are carried out at flow rates of only about 2 liter/minute or less. Scale-up of the untwisting tube design to flow rates higher than 2 liter/min has not been attempted. While reliability of such a device is unknown, it is expected to decrease with increasing scale.

Another group of prior art centrifuges to which the improvement of this invention may pertain are the designs of U.S. Pat. Nos. 4,086,924 and 4,300,717 which have a feeding system that includes a feed tube and accelerator as well as a centripetal pump or paring discs for discharge. While air often gets entrained in the liquid stream during the feed step, foaming of discharge liquids is controlled by use of the centripetal pump. These centrifuges, which are used exclusively for blood processing, are of single-use design and are quite small—flow rates do not exceed a few hundred milliliters/minute.

Some disk stack multi-use centrifuges have been designed which avoid air entrainment during the feeding step. They are typically referred to as “hermetic designs.” However, the resulting centrifuges are too mechanically complex for use in single-use centrifuge systems. Moreover, many of these designs require mechanical seals that are in contact with a process fluid path. This contacting must be avoided in the bioprocess industry because mechanical seals tend to shed particles into the fluid stream and those particles have been known to contaminate drug products. The present invention on the other hand uses a mechanical seal that only excludes air from the system and does not contact any process fluid.

Multi-use disc stack centrifuges typically discharge cells during rotation and the mechanisms used for discharge are too complex to be incorporated into single-use centrifuges. Also, the cells are often destroyed during discharge. Therefore multi-use stack designs are only used in bioprocess applications where viable, intact cells are not a requirement. The present invention on the other hand can be used to harvest intact, viable cells, as well as a centrate that is free of air and foaming problems.

U.S. Pat. No. 6,616,590 discloses a series of multi-use solid bowl centrifuges used in mammalian cell culture separations. While this design is capable of harvesting viable, intact cells, by using a low-shear feed accelerator that does not require a seal in a fluid path, it uses a feed tube and accelerator than can entrain air as well as a weir-type of centrate discharge. Thus there is a significant risk of centrate foaming.

The most relevant prior art to the present invention is found in the field of continuous and semi-continuous centrifuges that include a removable, presterilized fluid path insert enabling single-use operation.

Accordingly, the present invention overcomes the flow rate constraints of previous single use, pre-sterilized centrifuge systems, and provides a means of feeding and collecting liquid streams aseptically from rotating components while avoiding any air contamination or foaming problems.

### SUMMARY OF THE INVENTION

The present invention comprises apparatus and methods for centrifugal separation of cells in large-scale cell culture—i.e. batches larger than at least 100 liters, more commonly batches ranging from about 300 to 2000 liters in volume—using pre-sterilized, single-use fluid path components. The centrifuges of the present invention are of pre-sterilized, single-use design, and are capable of processing cell suspensions at flow rates in the range of about 3 to about 30 liters per minute, preferably about 7 to about 20 liters per minute. This flow capacity results in total run times in the range of about 2 to about 4 hours for a 2000 liter bioreactor batch harvest.

The devices of the present invention avoid the use of “untwisting” tubes to convey liquids to or from rotating components. Additionally, the devices do not have contacting-type seals in direct contact with process liquids.

By incorporating a sealing disc with flooded feed zone in combination with a centripetal pump for discharge, the present invention eliminates both sources of air entrainment and foam generation. Moreover, the invention uses a movable feed tube that enables the sealing disc and flooded feed zone to function with a simple, low-shear discharge approach for harvested cells. This sealing approach not only offers improved reliability and minimizes risk of contamination by both external agents and shedding from mechanical seals, but also minimizes the risk of leaks of process liquids.

### BRIEF DESCRIPTION OF THE DRAWINGS

FIG. 1 is a schematic view of a pre-sterilized, single use centrifuge system of the present invention during a feed cycle wherein only the pre-sterilized, single-use components of the system are shown, i.e. both rotating components and stationary support components have been omitted. In use the components outlined in a thin black line are stationary, while those outlined in thick solid or dotted lines rotate.

FIG. 2 is an expanded view of the connections among the following elements of FIG. 1: the inner feed tube, the outer feed tube, the centrate discharge tube, and the rigid upper flange of the rotating bowl.

FIG. 3 is a schematic view of the centrifuge system of FIG. 1 during a discharge cycle. The inner feed tube 1 has been extended downward to within close proximity of the bottom of the chamber which contains cell concentrate. As the inner feed tube 1 is moved down, the outer feed tube 3 remains stationary and the protective bellows 2 are compressed, maintaining sterility of the system.

FIG. 4 is a schematic view of an alternative centrifuge system in accordance with the present invention wherein the single use components are shown in black and permanent components are shown in gray.

FIG. 5 is a close-up view of the upper flange area of the centrifuge of FIG. 4, which shows a preferred method of sealing the flexible chamber material to the surface of the flange.

### DETAILED DESCRIPTION OF THE INVENTION

The present invention comprises apparatus and methods for centrifugal separation of cells in large-scale cell cultures—i.e. batches of about 2000 and more liters in volume. The centrifuges of the present invention are of pre-sterilized, single-use design and are capable of processing such cell suspensions at flow rates exceeding 20 liters per minute. This flow capacity enables total run times in the range of 2 to 3 hours for a 2000 liter bioreactor batch harvest. More preferably, the single-Use centrifuge systems are capable of processing about 300 to 2,000 liters of fluid while operating at a rate of about 3 to 30 liters per minute.

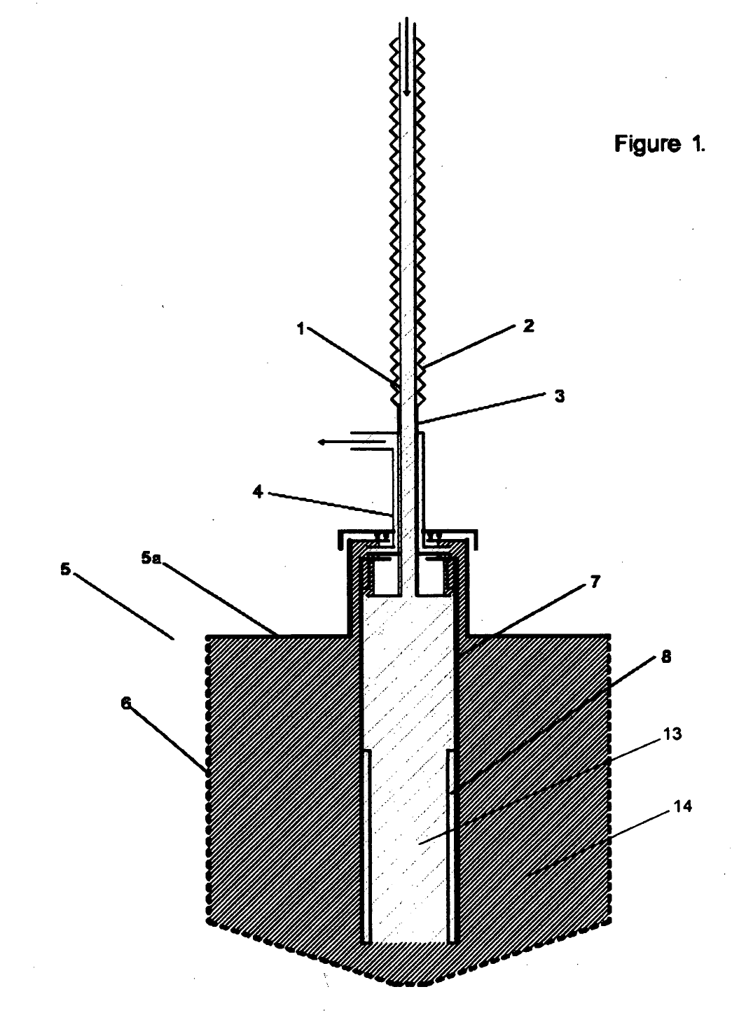

FIG. 1 shows a preferred embodiment of the present invention. FIG. 1 is a schematic view of a centrifuge system showing only the replaceable pre-sterilized, single-use components. Both rotating and stationary support components have been omitted for simplicity. The components shown in a thin line are stationary, while those in a thick line rotate. The components shown by solid thick lines are preferably formed by plastic molding, while those shown by dashed thick lines are preferably a flexible plastic film.

FIG. 1 shows an inner feed tube 1 sterilely connected to a source of a cell suspension, e.g. a bioreactor and suitable pump (not shown). The inner feed tube 1 passes thorough an outer feed tube 3 to which it is sealed by means of a flexible bellows 2. A centrate discharge tube 4 is disposed coaxially with respect to the outer feed tube 3, forming an annular discharge conduit. The exit of the centrate discharge tube 4 is sterilely connected to a centrate-receiving vessel (not shown). All of the components described thus far are shown in thin lines, denoting that they are stationary and are supported by a structure that is not shown in this Figure.

The pre-sterilized, single-use inner bowl 5 comprises a rigid upper flange 5a (thick solid line) and a flexible plastic liner 6 (thick dotted line). The flexible plastic liner 6 is completely supported by a rigid outer bowl (not shown) that is a permanent component of the centrifuge. The rigid upper flange 5a is attached to the upper rim of the rigid outer bowl, which serves to transmit torque to the entire rotating assembly. Within the inner bowl 5 is a cylindrical central core 7, which is attached to upper flange 5a by ribs that are not shown. The lower portion of cylindrical core 7 preferably contains one or more accelerator fins 8.

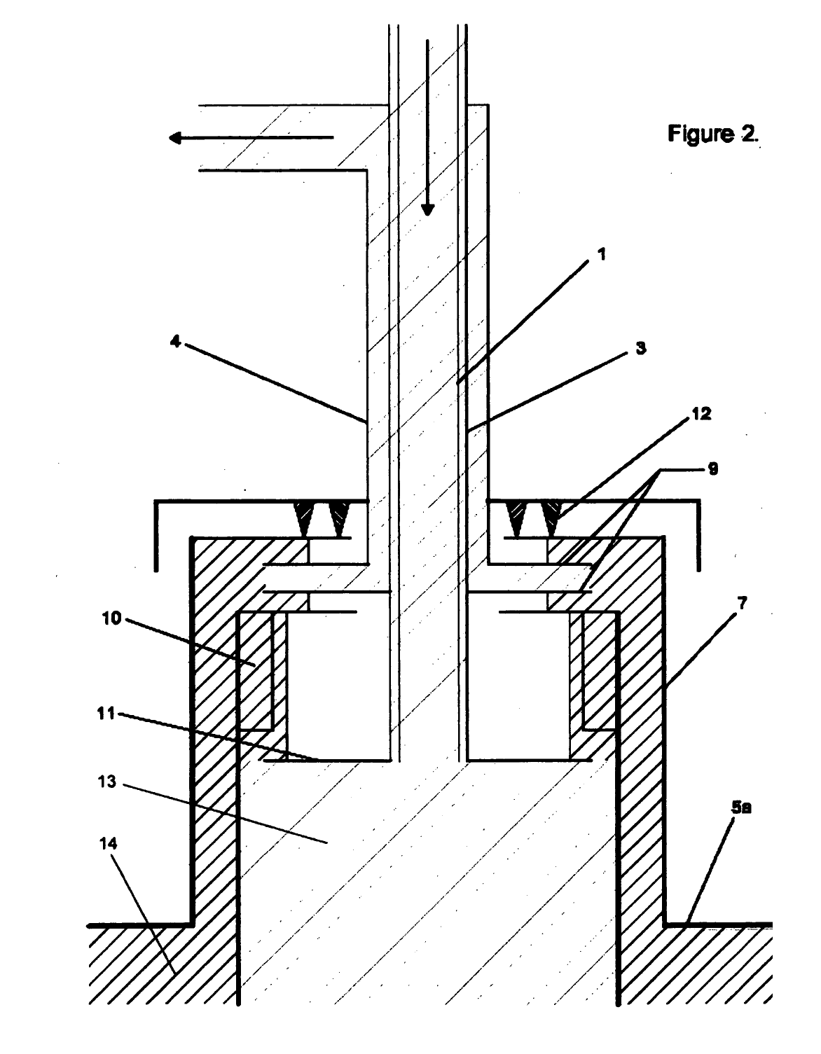

FIG. 2 shows details of the connections among the inner feed tube 1, the outer feed tube 3, the centrate discharge tube 4, and the rigid upper flange 5a of inner bowl 5. As shown, a set of paring discs 9 is attached to the outer feed tube 3 and the centrate discharge tube 4. Small accelerator fins 10 are located within the upper portion of central core 7. A hermetic liquid sealing flange 11 is located at the end of outer feed tube 3, and a contact-type rotating seal 12 is used to prevent ambient air from entering the sterile envelope. This rotating seal 12 is strictly a gas seal and does not come in contact with any process liquid. The rotating seal 12 is shown as a double lip seal, although a mechanical seal or another seal type may be used for this function.

The accelerator fins 10 work in conjunction with the liquid sealing flange 11 in the following manner. The first small volume of liquid that passes above the liquid sealing flange 11 is accelerated to bowl speed. This imparts to the liquid above the liquid sealing flange an increase in angular momentum relative to the unaccelerated liquid entering below the liquid sealing flange. This difference in angular momentum enables the establishment of a pressure difference between the upper and lower sides of the liquid sealing flange. The accelerator fins 10 and liquid sealing flange 11 enable operation of the system with a flooded feed zone while avoiding the presence of a contact-type rotating seal in liquid contact and the problems associated therewith, thereby enabling use of a non-contact hermetic seal that is suitable for use in pre-sterilized, single-use centrifuge systems.

During a feeding cycle, a feed suspension flows into the rotating bowl assembly through the inner feed tube 1. As the feed suspension enters the central core 7, it has not yet been accelerated to the angular velocity of the rotating bowl (denoted by lighter cross hatching 13 best seen in FIG. 1). As the feed suspension passes downward through the central core 7 toward the bottom of the bowl, it encounters the fins 8 that aid in accelerating the liquid up to bowl speed (denoted by darker cross-hatching 14 best seen in FIG. 1).

This invention is not limited to this design of feed accelerator. Alternatively, for example, feed acceleration could also be accomplished by fins projecting radially outward from the bottom of central core 7.

Centrate collects in the annular space between the upper flange of 5 and central core 7, flowing upward until encountering paring discs 9. The paring discs 9 are stationary components that collect the centrate without any air contact and discharge it under pressure, thus avoiding foaming. The paring discs 9 convert the kinetic energy of the rotating liquid to a pressure, serving to discharge centrate through discharge tube 4. The paring discs provide an improved means of centrate discharge, avoiding the possible shedding of particles into the liquid that occurs with mechanical seals in liquid contact, and the excessive foaming that often occurs with the weir approach to centrate discharge (whereby the centrate travels at a high velocity across an air gap and then impinges on a solid surface).

In the present invention the discharge of a cell concentrate is accomplished by momentarily stopping bowl rotation and then pumping out the cell concentrate that was formed. The rotating bowl 5 is sized so that its volumetric capacity for cell concentrate enables some batches to be processed in a single cycle. For the largest and most concentrated batches, a few operating cycles may be necessary. For example, if a 1000 liter bioreactor contains a cell culture batch that is 5% cells by volume, then the total cell concentrate to be discharged is 50 liters by volume. Thus a bowl of 25 liter volumetric capacity would have to be stopped once during the run to discharge cell concentrate and then discharged again at the end of the run. The range of volumetric bowl capacities that is compatible with the present invention is about 1 to 50 liters.

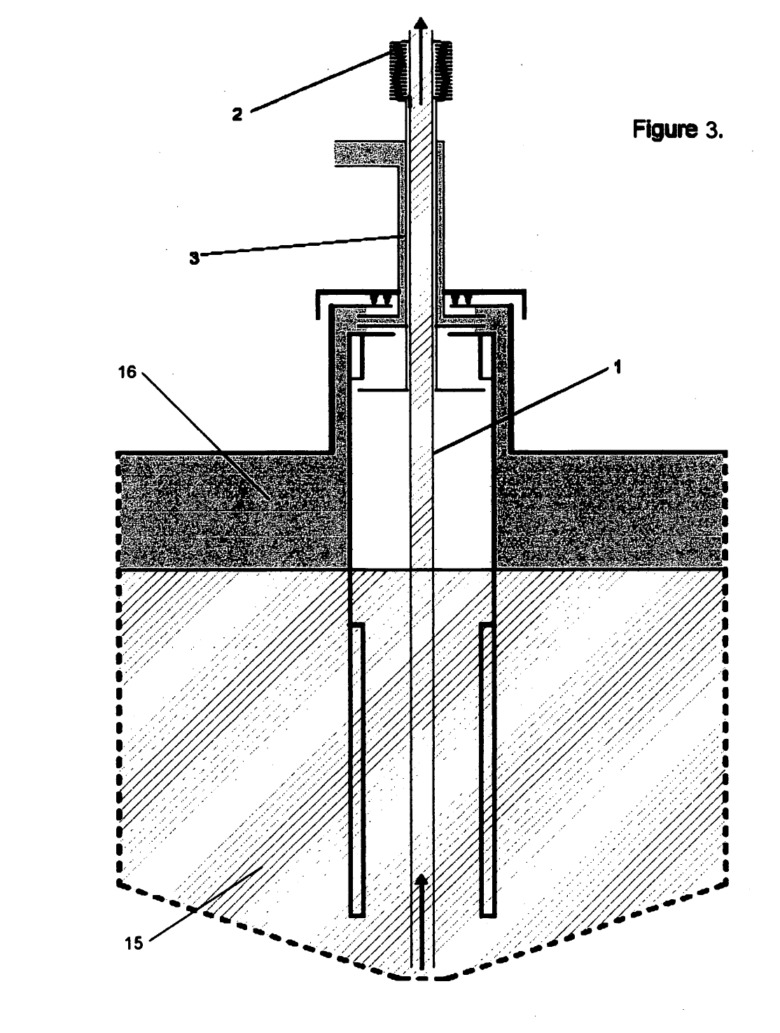

In FIG. 3, the centrifuge system is depicted at the start of a discharge cycle. The crosshatched area 15 denotes cell concentrate that is in the process of being discharged. The gray-shaded area 16 denotes cell-free centrate. As seen in FIG. 3, when the inner bowl 5 is filled to capacity, the cell concentrate does not reach the uppermost section of the bowl where the paring discs 9 and rotating seal 12 are located.

When the volumetric capacity of the inner bowl 5 is filled with concentrate, rotation is ceased. The inner feed tube 1 is moved downward, compressing the bellows 2, and passing through the outer feed tube 3, stopping just short of the bottom of the inner bowl 5. Then the cell concentrate is withdrawn by pumping it out through the inner feed tube 1. Appropriate valving (not shown) is used external to the centrifuge to direct the concentrate into a collection vessel (not shown). If the entire bioreactor batch has not yet been completely processed, then bowl rotation is resumed, followed by additional feed and discharge cycles until the batch has been processed to the extent desired.

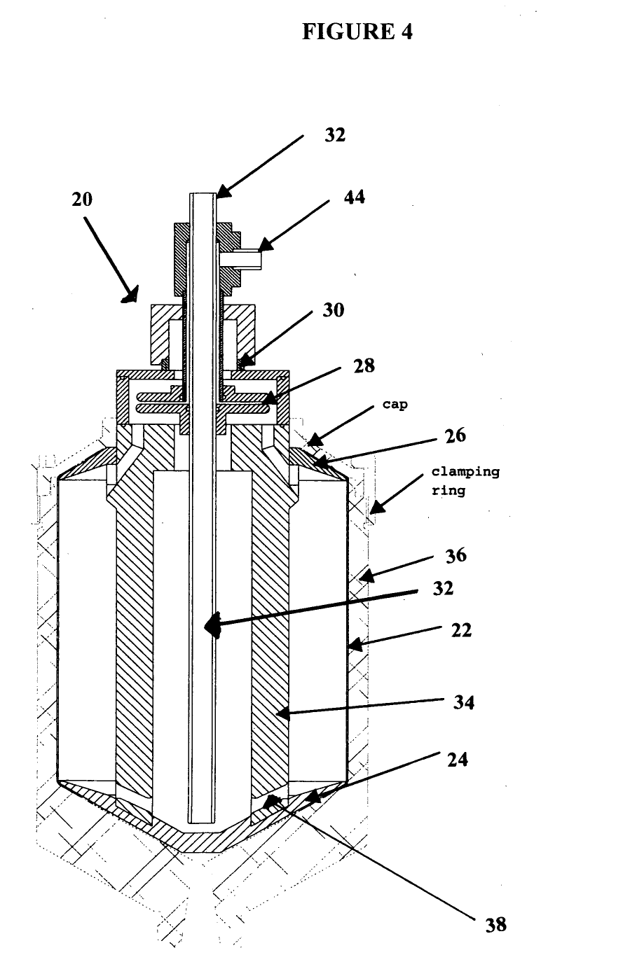

FIG. 4 discloses an improved alternative single use centrifuge structure 20 wherein the flexible plastic liner that extends to the bottom of the bowl in FIG. 1 is replaced by a flexible cylindrical liner 22, a lower flange 24 has been added and the flexible liner 22 is sealed to both an upper flange 26 and the lower flange 24. A centripetal pump 28 and a rotating mechanical seal 30 (also shown in FIGS. 1-3) are incorporated.

The upper flange 26, the core 34 and the lower flange 24 are preferably formed as a unitary structure to assist in maintaining the flexible liner 22 in place along the inside of a solid multiple-use bowl 36, thereby improving the flow of feed fluid to the outer chamber defined by the single use elements wherein particles of density higher than that of the liquid are captured by sedimentation. To assist in transfer of liquid from the feed tube 32 to the chamber defined by the flexible liner 22, multiple holes 38 may be provided through the core 34.

FIG. 4 shows a feed concentrate connection means 32 which includes a feed tube 33 that extends into the position shown in FIG. 3, close to the bottom of the structure. In this position the feed tube can fulfill both feed and discharge functions without needing to move the tube.

FIG. 4 further includes a centripetal pump 28 for centrate discharge through a centrate connection 44. When tested with a foaming medium, it did not generate foam.

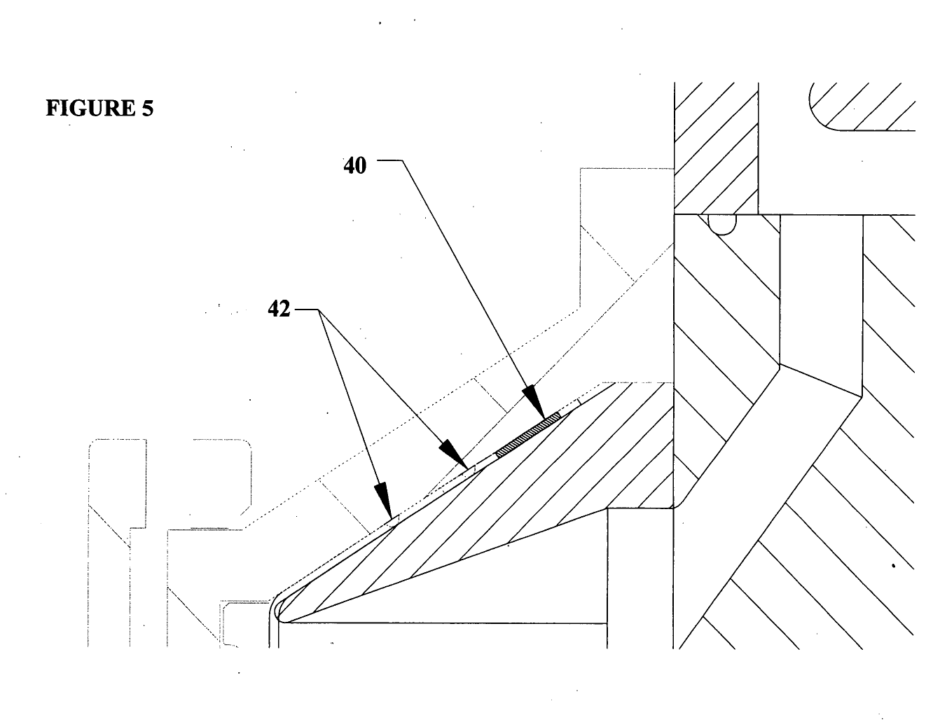

FIG. 5 shows a structure that provided improved sealing of the flexible liner to the upper and lower flanges. The flexible liner 22 may be a thermoplastic elastomer such as a polyurethane (TPU) or other stretchable, tough, non-tearing, bio-compatible polymer, while the upper and lower flanges may be fabricated from a rigid polymer such as polyetherimide, polycarbonate, or polysulfone. A thermal bonding attachment process is used to bond the dissimilar materials in the area shown in FIG. 5. The thermal bond is formed by pre-heating the flange material, placing the elastomeric polymer atop the heated flange, and applying heat and pressure to the elastomeric film at a temperature above its softening point.

The single-use components are pre-sterilized. During the transfer of these components from their protective packaging and installation into a centrifuge, the thermal bonds maintain sterility within the single-use chamber.

In addition to the thermal bond, sealing ridges or “nubbins” 42 are present on a metallic bowl cover 44 to compress the thermoplastic elastomeric film against the rigid upper flanges 26, forming an additional seal. The same compression seal is also utilized at the bottom of the bowl 36 to attach the thermoplastic elastomeric film against the rigid lower flanges 24. These compression seals isolate the thermal bonded areas from the hydrostatic pressure that develops during centrifugation when the chamber is filled with liquid. The combination of the thermal bond and the compression nubbin seals has been tested at 3000×g, which corresponds to a hydrostatic pressure of 97 psi at the bowl wall. In the test, a flexible TPU liner was used which was only 0.010 inch thick, yet the sealing means was completely effective and no leaks were observed.

The structure of FIGS. 4-5 does not require the hydrohermetic seal disc of FIGS. 1-3 and thus the elements that work in conjunction with the hydrohermetic seal—i.e. the upper and lower vanes and bellows—are not included.

The structure of FIGS. 4-5 has been prepared for use within a bowl that was 5.5 inches in diameter. At 2000×g it had a hydraulic capacity >7 liters/min and successfully separated mammalian cells to 99% efficiency at a rate of 3 liter/min.

What is claimed is:

1. A single use centrifuge system for centrifugal separation of cells in a cell culture batch, wherein cell suspensions are processed at a flow rate of about 3 to about 30 liters per minute.

2. The single use centrifuge system of claim 1, wherein the cell culture batch is larger than at least 100 liters.

3. The single use centrifuge system of claim 1, wherein the flow rate is about 7 to about 20 liters per minute.

4. The single use centrifuge system of claim 1, comprising a sealing disc with flooded feed zone in combination with a centripetal pump for discharge.

5. The single use centrifuge system of claim 1, which processes about 300 to 2,000 liters of fluid while operating at a rate of about 3 to 30 liters per minute.

6. The single use centrifuge system of claim 1, comprising an inner feed tube sterilely connected to a source of a cell suspension, wherein the inner feed tube passes thorough an outer feed tube to which it is sealed, and a centrate discharge tube disposed coaxially with respect to the outer feed tube, forming an annular discharge conduit.

7. The single use centrifuge system of claim 6, further comprising a centripetal pump and a rotating mechanical seal 30.

8. The single use centrifuge system of claim 6, further comprising an upper flange, a core, and a lower flange as a unitary structure, which structure maintains a flexible single use liner in place along the inside of a solid multiple-use bowl.

9. The single use centrifuge system of claim 8, wherein the upper flange, core and lower flange are prepared from a rigid polymer.

10. The single use centrifuge system of claim 8, wherein the core contains multiple holes extending therethrough to assist in transfer of liquid from the feed tube to a chamber defined by the flexible liner 22.

11. The single use centrifuge system of claim 1, comprising a flexible cylindrical liner, a upper flange, and a lower flange, wherein the flexible cylindrical liner is sealed to both the upper flange and the lower flange.

12. The single use centrifuge system of claim 10, wherein the flexible cylindrical liner is sealed to the upper flange and the lower flange by a combination of (i) thermal bonding and (ii) compression nubbins.

13. The single use centrifuge system of claim 1, wherein the single use components are pre-sterilized.

14. A single use centrifuge system for centrifugal separation of cells in a cell culture batch, wherein a flexible cylindrical liner is sealed to an upper element and the lower element by a combination of (i) thermal bonding and (ii) compression nubbins.

15. The single use centrifuge system of claim 14, wherein cell suspensions are processed at a flow rate of about 3 to about 30 liters per minute.
